(19) United States
(12) Patent Application Publication
(10) Pub. No.: US 2022/0031626 A1
(43) Pub. Date: Feb. 3, 2022

(54) Kind of Chinese medicine and microcapsule preparation method suitable for nasopharyngeal carcinoma tumor inhibitor

(71) Applicant: Haowei Ti, Fengqiu (CN)

(72) Inventors: Haowei Ti, Xinxiang (CN); Shiqi Mei, Shimen (CN); Donghai Mei, Shimen (CN)

(21) Appl. No.: 17/450,836

(22) Filed: Oct. 14, 2021

(51) Int. Cl. A61K 9/50; A61K 36/748; A61K 36/539; A61K 36/533; A61K 36/64; A61K 36/59; A61K 36/9066; A61K 36/47; A61K 36/634; A61K 36/346; A61K 36/54; A61K 36/288

(52) U.S. Cl. CPC A61K 9/5052; A61K 36/748; A61K 36/539; A61K 36/533; A61K 36/64; A61K 36/59; A61K 9/5036; A61K 36/47; A61K 36/634; A61K 36/346; A61K 36/54; A61K 36/288; A61K 9/5089; A61K 36/9066

(57) ABSTRACT

The invention relates to the technical field of traditional Chinese medicine, and discloses a method of traditional Chinese medicine and microcapsule preparation suitable for nasopharyngeal cancer tumor inhibitors. A traditional Chinese medicine suitable for nasopharyngeal cancer tumor inhibitors, including: Hedyotis Diffusa, Scutellaria Barbata, Arrowshaped Tinospora Root, Rehmanniae Radix Preparata, Radix Curcumae, Crotonis Fructus, Leonurus Artemisia, Ramulus Cinnamomi, Forsythiae Fructus, Taraxacum, Radix Platycodonis and White Vinegar. Among them, the raw materials for the weight ratio are as follows: 60 grams of Hedyotis Diffusa, 30 grams of Scutellaria Barbata, 9-12 grams of Arrowshaped Tinospora Root, 18 grams of Rehmanniae Radix Preparata, 19 grams of Radix Curcumae, 7.5 grams of Crotonis Fructus, 10 grams of Leonurus Artemisia, 7-10 grams of Ramulus Cinnamomi, 10 grams of Forsythiae Fructus, 15-30 grams of Taraxacum, 10 g of Radix Platycodonis, and 30 ml of White Vinegar.

### TECHNICAL FIELD

The invention relates to the technical field of traditional Chinese medicine, in particular to a method of traditional Chinese medicine and microcapsule preparation suitable for nasopharyngeal cancer tumor inhibitors.

### BACKGROUND TECHNIQUE

Conventional treatment of nasopharyngeal carcinoma in hospitals is intensity-modulated radiotherapy. In the initial treatment process, inhibitors are rarely used or directly enter the chemotherapy stage, which causes certain harm to the patient.

Radiation therapy is a kind of local treatment. Local control may have certain benefits for distant metastases, but doctors have not fully advocated strengthening systemic therapy to improve survival and reduce distant metastases. As far as the current systemic treatment is concerned, chemotherapy is mainly used, and drugs are rarely proposed to inhibit tumor development and metastasis.

In clinical treatment, the treatment focus is centered in the early stage, and the treatment cost is relatively high. In the preliminary treatment process, combined adjuvant chemotherapy or induction chemotherapy is mainly used, and it is difficult for ordinary families to bear the high cost.

The survival period of nasopharyngeal carcinoma is prolonged, and more and more patients have recurrence of nasopharyngeal after radiotherapy with radiation brain damage, recurrence after two or more courses of radiotherapy, and multiple distant metastases that have failed second-line treatment; another part of patients Recurrent nasopharyngeal carcinoma that failed standard treatment.

Radiochemical treatment was forced to interrupt radiotherapy due to the accumulation of side effects caused by non-specific sensitization, which affected the therapeutic effect.

In view of the toxic and side effects of radiotherapy and chemotherapy on the human body, traditional Chinese medicine can be combined in the treatment of advanced nasopharyngeal carcinoma to increase efficiency and reduce toxicity. Few doctors advocate the use of traditional Chinese medicine and Western medicine for the treatment of nasopharyngeal cancer. Traditional radiotherapy and chemotherapy, in addition to killing cancer cells, also kill normal cells, resulting in a decline in the patient's immunity. After radiotherapy and chemotherapy, the doctor blindly infuse the patient with nutrient solution, which will speed up the proliferation of cancer cells.

Conventional drugs for the treatment of nasopharyngeal cancer are relatively few, and the effect of targeted therapy is not very obvious, especially in the early stage of inhibition of drugs. The formulation of traditional Chinese medicine preparations is difficult to grasp and the shelf life is relatively short, so it cannot be put into clinical treatment well, nor can it be promoted on a large scale. For this reason, we have proposed a method of traditional Chinese medicine and microcapsule preparation suitable for nasopharyngeal cancer tumor inhibitors.

### INVENTION CONTENT

### (1) Technical Problems Solved

Aiming at the shortcomings of the prior art, the present invention provides a method of traditional Chinese medicine and microcapsule preparation suitable for nasopharyngeal cancer tumor inhibitors.

### (2) Technical Plan

In order to achieve the above objective, the present invention provides the following technical solutions: A Chinese medicine suitable for nasopharyngeal cancer tumor inhibitors, including: Hedyotis Diffusa, Scutellaria Barbata, Arrowshaped Tinospora Root, Rehmanniae Radix Preparata, Radix Curcumae, Crotonis Fructus, Leonurus Artemisia, Ramulus Cinnamomi, Forsythiae Fructus, Taraxacum, Radix Platycodonis and White Vinegar, including the following raw materials in parts by weight: Hedyotis Diffusa 60 grams, Scutellaria Barbata 30 grams, Arrowshaped Tinospora Root olive 9-12 grams, Rehmanniae Radix Preparata 18 grams, Radix Curcumae 19 grams, Crotonis Fructus 7.5 grams, Leonurus Artemisia 10 grams, Ramulus Cinnamomi 7-10 grams, Forsythiae Fructus 10 grams, Taraxacum 15-30 grams, Radix Platycodonis 10 g and White Vinegar 30 ml.

A preparation method of traditional Chinese medicine microcapsules suitable for nasopharyngeal cancer tumor inhibitors is characterized in that it comprises the following steps:

1) With milk protein as the main wall material and Artemisia annua starch as the auxiliary wall material, the auxiliary wall material accounts for 0-90% of the wall material. This preparation uses radix platycodonis oil as the core material;
2) The wall material is crushed to above 90 mesh and mixed evenly. Then, while stirring, slowly add the core material. After stirring and mixing uniformly, the formed core material is uniformly dispersed in the inhibitor under stirring. After the wall material is fully hydrated, it is homogenized by a homogenizer at 20-40 MPa for 2 to 4 times to obtain a stable emulsion. The microcapsule embedding process is completed, and the core material accounts for 55% of the mass of the microcapsule;
3) The emulsion uses high temperature heat treatment, aldehyde cross-linking and enzymatic exchange methods to solidify the wall material protein, so that the microcapsule wall is insoluble in water, and the core material is solid or semi-solid in water;
4) The curing liquid is spray-dried to become a free-flowing solid powder.
5) Microcapsules can be used as the main material, clinically made into pills, suspensions, and injections for treatment.

### (3) Beneficial Effects

Compared with the prior art, the present invention provides a traditional Chinese medicine and microcapsule preparation method suitable for nasopharyngeal cancer tumor inhibitors. It has the following beneficial effects:

1. This method is suitable for the preparation of traditional Chinese medicine and microcapsules for nasopharyngeal cancer tumor inhibitors. Due to the special shelf life and production process of traditional Chinese medicine preparations, many inconveniences are caused in the process of controlling fire and treating patients. The present invention also adopts microcapsule technology for traditional Chinese medicine formulations, which can protect the effective ingredients and prolong the effective period, especially the effect of traditional Chinese medicine decoctions is more obvious.
2. This method is suitable for traditional Chinese medicine and microcapsule preparation methods for nasopharyngeal cancer tumor inhibitors. The release of the active ingredients in the controlled release microcapsules of microcapsule preparations can be controlled by immediate release, delayed, timed-release or long-acting release, etc. The microcapsule preparation reduces toxicity, is safe and environmentally friendly, and can further improve the efficacy of the drug.

### Specific Implementation

The technical solutions in the embodiments of the present invention will be clearly and completely described below in conjunction with the embodiments of the present invention. Obviously, the described embodiments are only a part of the embodiments of the present invention, rather than all the embodiments. Based on the embodiments of the present invention, all other embodiments obtained by those of ordinary skill in the art without creative work shall fall within the protection scope of the present invention.

Example 1: A Chinese medicine suitable for nasopharyngeal cancer tumor inhibitors, including: Hedyotis Diffusa, Scutellaria Barbata, Arrowshaped Tinospora Root, Rehmanniae Radix Preparata, Radix Curcumae, Crotonis Fructus, Leonurus Artemisia, Ramulus Cinnamomi, Forsythiae Fructus, Taraxacum, Radix Platycodonis and White Vinegar.

Hedyotis Diffusa: clearing away heat and detoxifying; eliminating dampness, controlling lung heat, asthma and cough; sore throat; intestinal carbuncle; furuncle swelling and sore; venomous snake bite; hot shower astringent pain; edema; dysentery; enteritis;

Scutellaria Barbata: pungent, bitter, cold, return to the lung, liver, kidney meridian, function and indications, clearing away heat and detoxification, removing blood stasis and diuresis, used for boils, swelling and toxin, sore throat, snake bite, falling pain, edema, jaundice.

Rehmanniae Radix Preparata: heat-clearing and promoting fluid, cooling blood, hemostasis, used for yin damage caused by fever, crimson tongue, polydipsia, spotted rash, vomiting blood, bleeding, swelling and sore throat.

Arrowshaped Tinospora Root: the dried root tuber of the tetragonaceae plant cyclobalanopsis or arrowshaped tinospora root. It is excavated in autumn and winter to remove the fibrous roots, wash, and dry. Used for sore throat, carbuncle and furuncle, diarrhea, dysentery, abdominal pain.

Radix Curcumae: promoting qi to remove blood stasis, clearing the heart and relieving depression, promoting gallbladder and relieving jaundice. It is used for amenorrhea, dysmenorrhea, chest and abdomen pain, tingling, fever, dizziness, epilepsy, jaundice and red urine.

Crotonis Fructus: Relieving cold accumulation, clearing the orifice, expelling phlegm, running water, killing insects, treating cold stagnation, fullness of chest and abdomen, acute pain, hematochezia, phlegm addiction, diarrhea, edema. External use to treat throat wind, throat numbness, malignant sores, scabies.

Leonurus Artemisia: Activating blood to regulate menstruation, diuresis and swelling, clearing away heat and detoxification, used for irregular menstruation, dysmenorrhea, amenorrhea, inexhaustible lochia, edema, oliguria, sore swelling and poison.

Ramulus Cinnamomi: sweats and relieves muscles, warms the meridians, helps yang and qi, calms down the qi. Commonly used for cold, cold, abdominal pain, blood-cold amenorrhea, arthralgia, phlegm, edema, palpitations, etc.

Forsythiae Fructus: wild vegetable oil, the oil content of forsythia suspensa seeds is 25%-33%. Seed oil contains colloids, has good volatility, and is rich in oleic acid and linoleic acid that are easily absorbed and digested by the human body.

Taraxacum: Attending to treat women's breast carbuncle, boiled in water, drink and seal the carbuncle. It detoxifies food, clears away heat and toxins, dissolves food toxins, reduces malignant swelling, tuberculosis, and boils.

Radix Platycodonis: promote lung, relieve throat, expectorant, drain pus. It is used for coughing with excessive sputum, poor chest tightness, sore throat, dull voice, lung carbuncle and vomiting pus, sore pus is not ulcerated.

White Vinegar: disperses blood stasis and dissipates accumulation; hemostasis; an roundworm; detoxification. It mainly treats postpartum haemorrhage; syndrome accumulation; vomiting blood; epistaxis; blood in the stool;

Among them, raw materials with the following proportions by weight: 60 grams of Hedyotis Diffusa, 30 grams of Scutellaria Barbata, 9-12 grams of Arrowshaped Tinospora Root, 18 grams of Rehmanniae Radix Preparata, 19 grams of Radix Curcumae, 7.5 grams of Crotonis Fructus, 10 grams of Leonurus Artemisia, 7-10 grams of Rumulus Cinnamomi, 10 grams of Forsythiae Fructus, 15-30 grams of Taraxacum, 10 g of Radix Platycodonis , and 30 ml of White Vinegar.

A preparation method of traditional Chinese medicine microcapsules suitable for nasopharyngeal cancer tumor inhibitors is characterized in that it comprises the following steps:

1) It uses milk protein as the main wall material, and Artemisia annua starch as the auxiliary wall material, and the auxiliary wall material accounts for 0-90% of the wall material; it uses radix platycodonis as the core material. Radix Platycodonis: It has the effects of promoting lungs, pharyngeal relief, expectorating, and purging pus. It can be used for coughing with excessive phlegm, chest tightness, dull sore throat, and lung carbuncle and vomiting pus;

2) The wall material is crushed to above 90 mesh and mixed evenly. Then, while stirring, slowly add the core material. After stirring and mixing uniformly, the core material will be formed. Disperse uniformly in the inhibitor under stirring, and after the wall material is fully hydrated, it is homogenized by a homogenizer at 20-40 MPa for 2 to 4 times to obtain a stable emulsion. Complete the microcapsule embedding process, the core material accounts for 55% of the mass of the microcapsule;

3) The latex liquid uses high temperature heat treatment, aldehyde cross-linking and enzymatic exchange methods to solidify the wall material protein, so that the microcapsule wall is insoluble in water, and the core material is solid or semi-solid in water;

4) The curing liquid is spray-dried to become a free-flowing solid powder.

5) Microcapsules can be used as the main material, clinically made into pills, suspensions, and injections for treatment.

Example 2: A traditional Chinese medicine suitable for nasopharyngeal cancer tumor inhibitors, including: Hedyotis Diffusa, Scutellaria Barbata, Arrowshaped Tinospora Root, Rehmanniae Radix Preparata, Radix Curcumae, Crotonis Fructus, Leonurus Artemisia, Ramulus Cinnamomi, Forsythiae Fructus, Taraxacum, Radix Platycodonis, and White Vinegar.

Hedyotis Diffusa: clearing away heat and detoxifying; eliminating dampness, controlling lung heat, asthma and cough; sore throat; intestinal carbuncle; furuncle swelling and sore; venomous snake bite; hot shower astringent pain; edema; dysentery; enteritis;

Scutellaria Barbata: pungent, bitter, cold, returned to the lung, liver, and kidney meridians. Functions and Indications: clearing heat and detoxification, removing blood stasis and diuresis, used for boils, swelling and toxin, sore throat, snake bite, swelling pain, edema, jaundice.

Rehmanniae Radix Preparata: clearing away heat and producing body fluid, cooling blood, and stopping bleeding. It is used for yin injury caused by fever, crimson tongue, polydipsia, spotted rash, vomiting blood, bleeding, throat swelling and pain.

Arrowshaped Tinospora Root: the dried root tuber of the tetragonaceae plant cyclobalanopsis or arrowshaped tinospora root. It is excavated in autumn and winter to remove the fibrous roots, wash, and dry. Used for sore throat, carbuncle and furuncle, diarrhea, dysentery, abdominal pain.

Radix Curcumae: promoting qi to remove blood stasis, clearing the heart and relieving depression, promoting gallbladder and relieving jaundice. It is used for amenorrhea, dysmenorrhea, chest and abdomen pain, tingling, fever, dizziness, epilepsy, jaundice and red urine.

Crotonis Fructus: Reducing cold accumulation, clearing the orifice, removing phlegm, running water, killing insects. It cures cold stagnation, fullness of chest and abdomen, acute pain, hematochezia, phlegm addiction, diarrhea, and edema. It is used externally to treat throat wind, throat numbness, malignant sores and scabies.

Leonurus Artemisia: Activating blood to regulate menstruation, diuresis and swelling, clearing away heat and detoxification. It is used for irregular menstruation, dysmenorrhea, amenorrhea, inexhaustible lochia, edema, oliguria, sores, swelling and poisoning.

Rumulus Cinnamomi: It has the effects of sweating and relieving muscles, warming the meridians, promoting yang and transforming qi, and calming and lowering qi. It is often used for cold, cold, abdominal pain, blood-cold amenorrhea, arthralgia, phlegm, edema, heart palpitations, etc.

Forsythiae Fructus: wild vegetable oil, the oil content of forsythia suspensa seeds is 25%-33%. Seed oil contains colloids, has good volatility, and is rich in oleic acid and linoleic acid that are easily absorbed and digested by the human body.

Taraxacum: Attending to treat women's breast carbuncle, boiled in water, drink and seal the carbuncle. It detoxifies food, dissipates stagnant qi, clears away heat and toxins, dissolves food toxins, eliminates malignant swelling, tuberculosis, and boils.

Radix Platycodonis: promote lung, relieve throat, expectorant, drain pus. It is used for coughing with excessive sputum, poor chest tightness, sore throat, dull voice, lung carbuncle and vomiting pus, sore pus is not ulcerated.

White Vinegar: dissipate blood stasis and dissipate accumulation; stop bleeding; relieve roundworm; detoxify, mainly postpartum haemorrhage; syndrome accumulation; vomiting blood; epistaxis; blood in the stool; worm accumulation; abdominal pain; fish meat and vegetable poison;

Among them, raw materials with the following proportions by weight: Hedyotis Diffusa 60 grams, Scutellaria Barbata 30 grams, Arrowshaped Tinospora Root olive 10 grams, Rehmanniae Radix Preparata 18 grams, Radix Curcumae 19 grams, Crotonis Fructus 7.5 grams, Leonurus Artemisia 10 grams, Rumulus Cinnamomi 8 g, 10 g Forsythiae Fructus, 20 g Taraxacum, 10 g Radix Platycodonis and 30 ml White Vinegar.

A preparation method of traditional Chinese medicine microcapsules suitable for nasopharyngeal cancer tumor inhibitors is characterized in that it comprises the following steps:

1)It uses milk protein as the main wall material, and Artemisia annua starch as the auxiliary wall material, and the auxiliary wall material accounts for 50% of the wall material by mass; it uses radix platycodonis as the core material. Radix Platycodonis: It has the effects of promoting lungs, pharyngeal relief, expectorating, and purging pus. It can be used for coughing with excessive phlegm, chest tightness, dull sore throat, and lung carbuncle and vomiting pus;

2) Crush the wall material to above 90 mesh, mix well, and then slowly add the core material under stirring. After stirring and mixing uniformly, the formed core material is uniformly dispersed in the inhibitor under stirring. After the wall material is fully hydrated, it is homogenized by a homogenizer at 20-40 MPa for 2 to 4 times to obtain a stable emulsion. Complete the microcapsule embedding process, the core material accounts for 55% of the mass of the microcapsule;

3) It uses high temperature heat treatment, aldehyde cross-linking and enzymatic exchange methods to solidify the wall material protein, so that the microcapsule wall is insoluble in water. It is a solid or semi-solid capsule film covering the core material in water;

4) The curing liquid is spray-dried to become a free-flowing solid powder.

5) Microcapsules can be used as the main material, clinically made into pills, suspensions, and injections for treatment.

Example 3: A Chinese medicine suitable for nasopharyngeal cancer tumor inhibitors, including: Hedyotis Diffusa, Scutellaria Barbata, Arrowshaped Tinospora Root, Rehmanniae Radix Preparata, Radix Curcumae, Crotonis Fructus, Leonurus Artemisia, Ramulus Cinnamomi, Forsythiae Fructus, Taraxacum, Radix Platycodonis, and White Vinegar.

Hedyotis Diffusa: clearing away heat and detoxifying; eliminating dampness, controlling lung heat, asthma and cough; sore throat; intestinal carbuncle; furuncle swelling and sore; venomous snake bite; hot shower astringent pain; edema; dysentery; enteritis;

Scutellaria Barbata: pungent, bitter, cold, returned to the lung, liver, and kidney meridians. Its function is to clear away heat and detoxify, remove blood stasis and diuresis. It is used for boils, swelling and poisoning, sore throat, poisonous snake bites, swelling pain, edema, and jaundice.

Rehmanniae Radix Preparata: clearing away heat and producing body fluid, cooling blood, and stopping bleeding. It is used for yin injury caused by fever, crimson tongue, polydipsia, spotted rash, vomiting blood, bleeding, throat swelling and pain.

Arrowshaped Tinospora Root: the dried root tuber of the tetragonaceae plant cyclobalanopsis or arrowshaped tinospora root. It is excavated in autumn and winter to remove the fibrous roots, wash, and dry. Used for sore throat, carbuncle and furuncle, diarrhea, dysentery, abdominal pain.

Radix Curcumae: promoting qi to remove blood stasis, clearing the heart and relieving depression, promoting gallbladder and relieving jaundice. It is used for amenorrhea, dysmenorrhea, chest and abdomen pain, tingling, fever, dizziness, epilepsy, jaundice and red urine.

Crotonis Fructus: Reducing cold accumulation, clearing the orifice, removing phlegm, running water, killing insects. It cures cold stagnation, fullness of chest and abdomen, acute pain, hematochezia, phlegm addiction, diarrhea, and edema. It is used externally to treat throat wind, throat numbness, malignant sores and scabies.

Leonurus Artemisia: Activating blood to regulate menstruation, diuresis and swelling, clearing away heat and detoxification. It is used for irregular menstruation, dysmenorrhea, amenorrhea, inexhaustible lochia, edema, oliguria, sores, swelling and poisoning.

Rumulus Cinnamomi: It has the effects of sweating and relieving muscles, warming the meridians, promoting yang and transforming qi, and calming and lowering qi. It is often used for colds, colds, abdominal pain, blood-cold amenorrhea, arthralgia, phlegm, edema, heart palpitations, etc.

Forsythiae Fructus: It is a wild vegetable oil, and the oil content of Forsythiae Fructus seeds is 25%-33%. Seed oil contains gums, has good volatility, and has good development potential. Oil is rich in oleic acid and linoleic acid, which are easily absorbed and digested by the body.

Taraxacum: Attending to treat women's breast carbuncle, boiled juice and seal it to eliminate, detoxify food, dissipate stagnant qi, clear heat toxin, reduce food poison, reduce malignant swelling, tuberculosis, and boil.

Radix Platycodonis: promote lung, relieve throat, expectorant, drain pus. It is used for coughing with excessive sputum, poor chest tightness, sore throat, dull voice, lung carbuncle and vomiting pus, sore pus is not ulcerated.

White Vinegar: dissipating blood stasis and dissipating accumulation; stopping bleeding; relieving roundworms; it detoxifies, the main postpartum blood halo; the accumulation of symptoms; vomiting blood; blemishes; blood in the stool;

Among them, raw materials with the following proportions by weight: Hedyotis Diffusa 60 grams, Scutellaria Barbata 30 grams, Arrowshaped Tinospora Root olive 11 grams, Rehmanniae Radix Preparata 18 grams, Radix Curcumae 19 grams, Crotonis Fructus 7.5 grams, Leonurus Artemisia 10 grams, Rumulus Cinnamomi 9 grams, 10 grams of Forsythiae Fructus, 22 grams of Taraxacum, 10 grams of Radix Platycodonis and 30 ml of White Vinegar.

A preparation method of traditional Chinese medicine microcapsules suitable for nasopharyngeal cancer tumor inhibitors is characterized in that it comprises the following steps:

1) It uses milk protein as the main wall material and Artemisia annua starch as the auxiliary wall material. The auxiliary wall material accounts for 60% of the wall material mass percentage; it is made of radix platycodonis as the core material. Radix Platycodonis: It has the effects of promoting lungs, pharyngeal relief, expectorating, and purging pus. It can be used for coughing with excessive phlegm, chest tightness, dull sore throat, and lung carbuncle and vomiting pus;

2) Crush the wall material to above 90 mesh, mix well, and then slowly add the core material under stirring. After stirring and mixing uniformly, the formed core material is uniformly dispersed in the inhibitor under stirring. After the wall material is fully hydrated, it is homogenized by a homogenizer at 20-40 MPa for 2 to 4 times to obtain a stable emulsion. Complete the microcapsule embedding process, the core material accounts for 55% of the mass of the microcapsule;

3) It uses high temperature heat treatment, aldehyde cross-linking and enzymatic exchange methods to solidify the wall material protein, so that the microcapsule wall is insoluble in water, and the core material is solid or semi-solid in water;

4) The curing liquid is spray-dried to become a free-flowing solid powder.

5) Microcapsules can be used as the main material, clinically made into pills, suspensions, and injections for treatment.

Example 4: A Chinese medicine suitable for nasopharyngeal cancer tumor inhibitors, including: Hedyotis Diffusa, Scutellaria Barbata, Arrowshaped Tinospora Root, Rehmanniae Radix Preparata, Radix Curcumae, Crotonis Fructus, Leonurus Artemisia, Ramulus Cinnamomi, Forsythiae Fructus, Taraxacum, Radix Platycodonis, and White Vinegar.

Hedyotis Diffusa: clearing away heat and detoxifying; eliminating dampness, controlling lung heat, asthma and cough; sore throat; intestinal carbuncle; furuncle swelling and sore; venomous snake bite; hot shower astringent pain; edema; dysentery; enteritis;

Scutellaria Barbata: pungent, bitter, cold, returned to the lung, liver, and kidney meridians. Its function is to clear away heat and detoxify, remove blood stasis and diuresis. It is used for boils, swelling and poisoning, sore throat, poisonous snake bites, swelling pain, edema, and jaundice.

Rehmanniae Radix Preparata: clearing away heat and producing body fluid, cooling blood, and stopping bleeding. It is used to injure the yin due to fever, crimson tongue, polydipsia, spotted rash, vomiting blood, bleeding, throat swelling and pain.

Arrowshaped Tinospora Root: the dried root tuber of the tetragonaceae plant cyclobalanopsis or arrowshaped tinospora root. It is excavated in autumn and winter to remove the fibrous roots, wash, and dry. Used for sore throat, carbuncle and furuncle, diarrhea, dysentery, abdominal pain.

Radix Curcumae: promoting qi to remove blood stasis, clearing the heart and relieving depression, promoting gallbladder and relieving jaundice. It is used for amenorrhea, dysmenorrhea, chest and abdomen pain, tingling, fever, dizziness, epilepsy, jaundice and red urine.

Crotonis Fructus: Reducing cold accumulation, clearing the orifice, removing phlegm, running water, killing insects. It cures cold stagnation, fullness of chest and abdomen, acute pain, hematochezia, phlegm addiction, diarrhea, and edema. It is used externally to treat throat wind, throat numbness, malignant sores and scabies.

Leonurus Artemisia: Activating blood to regulate menstruation, diuresis and swelling, clearing away heat and detoxification. It is used for irregular menstruation, dysmenorrhea, amenorrhea, inexhaustible lochia, edema, oliguria, sores, swelling and poisoning.

Rumulus Cinnamomi: It has the effects of sweating and relieving muscles, warming the meridians, promoting yang and transforming qi, and calming and lowering qi. It is often used for colds, colds, abdominal pain, blood-cold amenorrhea, arthralgia, phlegm, edema, heart palpitations, etc.

Forsythiae Fructus:It is a wild vegetable oil. Forsythiae Fructus seeds has an oil content of 25%-33%. The seed oil contains gums, has good volatility, and has good development potential. It is rich in oleic acid and linoleic acid, which are easily absorbed and digested by the body.

Taraxacum: Attending to treat women's breast carbuncle, boiled in water, drink and seal the carbuncle. It detoxifies food, dissipates stagnant qi, clears away heat and toxins, dissolves food toxins, eliminates malignant swelling, tuberculosis, and boils.

Radix Platycodonis: promote lung, relieve throat, expectorant, drain pus. It is used for coughing with excessive sputum, poor chest tightness, sore throat, dull voice, lung carbuncle and vomiting pus, sore pus is not ulcerated.

White Vinegar: dissipating blood stasis and dissipating accumulation; stopping bleeding; relieving roundworms; it detoxifies, the main postpartum blood halo; the accumulation of symptoms; vomiting blood; blemishes; blood in the stool;

Among them, raw materials with the following proportions by weight: Hedyotis Diffusa 60 grams, Scutellaria Barbata 30 grams, Arrowshaped Tinospora Root 11 grams, Rehmanniae Radix Preparata 18 grams, Radix Curcumae 19 grams, Crotonis Fructus 7.5 grams, Leonurus Artemisia 10 grams, Rumulus Cinnamomi 10 grams, 10 grams of Forsythiae Fructus, 27 grams of Taraxacum, 10 grams of Radix Platycodonis and 30 ml of White Vinegar.

A preparation method of traditional Chinese medicine microcapsules suitable for nasopharyngeal cancer tumor inhibitors is characterized in that it comprises the following steps:

1)It uses milk protein as the main wall material and Artemisia annua starch as the auxiliary wall material. The auxiliary wall material accounts for 80% of the wall material mass percentage; it uses radix platycodonis oil as the core material. Radix Platycodonis: It has the effects of promoting lungs, improving throat, expelling phlegm, and draining pus. It is used for coughing with excessive phlegm, poor chest tightness, dumb sore throat, and pulmonary carbuncle vomiting pus;

2) The wall material is crushed to above 90 mesh and mixed evenly. Then, while stirring, slowly add the core material. After stirring and mixing uniformly, the formed core material is uniformly dispersed in the inhibitor under stirring. After the wall material is fully hydrated, it is homogenized by a homogenizer at 20-40 MPa for 2 to 4 times to obtain a stable emulsion. Complete the microcapsule embedding process, the core material accounts for 55% of the mass of the microcapsule;

3) It uses high temperature heat treatment, aldehyde cross-linking and enzymatic exchange methods to solidify the wall material protein, so that the microcapsule wall is insoluble in water, and the core material is solid or semi-solid in water;

4) The curing liquid is spray-dried to become a free-flowing solid powder.

5) Microcapsules can be used as the main material, clinically made into pills, suspensions, and injections for treatment.

Example 5: A Chinese medicine suitable for nasopharyngeal cancer tumor inhibitors, including: Hedyotis Diffusa, Scutellaria Barbata, Arrowshaped Tinospora Root, Rehmanniae Radix Preparata, Radix Curcumae, Crotonis Fructus, Leonurus Artemisia, Ramulus Cinnamomi, Forsythiae Fructus, Taraxacum, Radix Platycodonis, and White Vinegar.

Hedyotis Diffusa: clearing away heat and detoxifying; eliminating dampness, controlling lung heat, asthma and cough; sore throat; intestinal carbuncle; furuncle swelling and sore; venomous snake bite; hot shower astringent pain; edema; dysentery; enteritis;

Scutellaria Barbata: pungent, bitter, cold, returned to the lung, liver, and kidney meridians. Its function is to clear away heat and detoxify, remove blood stasis and diuresis. It is used for boils, swelling and poisoning, sore throat, poisonous snake bites, swelling pain, edema, and jaundice.

Rehmanniae Radix Preparata: clearing away heat and producing body fluid, cooling blood, and stopping bleeding. It is used to injure the yin due to fever, crimson tongue, polydipsia, spotted rash, vomiting blood, bleeding, throat swelling and pain.

Arrowshaped Tinospora Root: the dried root tuber of the tetragonaceae plant cyclobalanopsis or arrowshaped tinospora root. It is excavated in autumn and winter to remove the fibrous roots, wash, and dry. Used for sore throat, carbuncle and furuncle, diarrhea, dysentery, abdominal pain.

Radix Curcumae: promoting qi to remove blood stasis, clearing the heart and relieving depression, promoting gallbladder and relieving jaundice. It is used for amenorrhea, dysmenorrhea, chest and abdomen pain, tingling, fever, dizziness, epilepsy, jaundice and red urine.

Crotonis Fructus: Reducing cold accumulation, clearing the orifice, removing phlegm, running water, killing insects. It cures cold stagnation, fullness of chest and abdomen, acute pain, hematochezia, phlegm addiction, diarrhea, and edema. It is used externally to treat throat wind, throat numbness, malignant sores and scabies.

Leonurus Artemisia: Activating blood to regulate menstruation, diuresis and swelling, clearing away heat and detoxification. It is used for irregular menstruation, dysmenorrhea, amenorrhea, inexhaustible lochia, edema, oliguria, sores, swelling and poisoning.

Rumulus Cinnamomi: It has the effects of sweating and relieving muscles, warming the meridians, promoting yang and transforming qi, and calming and lowering qi. It is often used for cold, cold, abdominal pain, blood-cold amenorrhea, arthralgia, phlegm, edema, heart palpitations, etc.

Forsythiae Fructus: It is a wild vegetable oil. Forsythiae Fructus seeds has an oil content of 25%-33%. The seed oil contains gums, has good volatility, and has good development potential. It is rich in oleic acid and linoleic acid, which are easily absorbed and digested by the body.

Taraxacum: Attending to treat women's breast carbuncle, boiled juice and seal it to eliminate, detoxify food, dissipate stagnant qi, clear heat toxin, reduce food poison, reduce malignant swelling, tuberculosis, and boil.

Radix Platycodonis: promote lung, relieve throat, expectorant, drain pus. It is used for coughing with excessive sputum, poor chest tightness, sore throat, dull voice, lung carbuncle and vomiting pus, sore pus is not ulcerated.

White Vinegar: dissipating blood stasis and dissipating accumulation; stopping bleeding; relieving roundworms; it detoxifies, the main postpartum blood halo; the accumulation of symptoms; vomiting blood; blemishes; blood in the stool;

Among them, raw materials with the following proportions by weight: Hedyotis Diffusa 60 g, Scutellaria Barbata 30 g, Arrowshaped Tinospora Root olive 9 g, Rehmanniae Radix Preparata 18 g, Radix Curcumae 19 g, Crotonis Fructus 7.5 g, Leonurus Artemisia 10 g, Rumulus Cinnamomi 7 grams, 10 grams of Forsythiae Fructus, 30 grams of Taraxacum, 10 grams of Radix Platycodonis and 30 ml of White Vinegar.

A preparation method of traditional Chinese medicine microcapsules suitable for nasopharyngeal cancer tumor inhibitors is characterized in that it comprises the following steps:

1) It uses milk protein as the main wall material and Artemisia annua starch as the auxiliary wall material. The auxiliary wall material accounts for 90% of the wall material mass percentage; it is made of radix platycodonis as the core material. Radix Platycodonis: It has the effects of promoting lungs, pharyngeal relief, expectorating, and purging pus. It can be used for coughing with excessive phlegm, chest tightness, dull sore throat, and lung carbuncle and vomiting pus;

2) The wall material is crushed to above 90 mesh and mixed evenly. Then, while stirring, slowly add the core material. After stirring and mixing uniformly, the formed core material is uniformly dispersed in the inhibitor under stirring. After the wall material is fully hydrated, it is homogenized by a homogenizer at 20-40 MPa for 2 to 4 times to obtain a stable emulsion. Complete the microcapsule embedding process, the core material accounts for 55% of the mass of the microcapsule;

3) It uses high temperature heat treatment, aldehyde cross-linking and enzymatic exchange methods to solidify the wall material protein, so that the microcapsule wall is insoluble in water, and the core material is solid or semi-solid in water;

4) The curing liquid is spray-dried to become a free-flowing solid powder.

5) Microcapsules can be used as the main material to make pills, suspensions, and injections for clinical treatment;

Although the embodiments of the present invention have been shown and described, for those of ordinary skill in the art, it will be understood that various changes, modifications, replacements and variants can be made to these embodiments without departing from the principle and spirit of the present invention. The scope of the present invention is defined by the appended claims and their equivalents.

1. A Chinese medicine suitable for nasopharyngeal cancer tumor inhibitors, which is characterized in that it comprises: Hedyotis Diffusa, Scutellaria Barbata, Arrowshaped Tinospora Root, Rehmanniae Radix Preparata, Radix Curcumae, Crotonis Fructus, Leonurus Artemisia, Ramulus Cinnamomi, Forsythiae Fructus, Taraxacum, Radix Platycodonis and White Vinegaris and White Vinegar. Among them, the raw materials for the weight ratio are as follows: 60 grams of Hedyotis Diffusa, 30 grams of Scutellaria Barbata, 9-12 grams of Arrowshaped Tinospora Root, 18 grams of Rehmanniae Radix Preparata, 19 grams of Radix Curcumae, 7.5 grams of Crotonis Fructus, 10 grams of Leonurus Artemisia, 7-10 grams of Ramulus Cinnamomi, 10 grams of Forsythiae Fructus, 15-30 grams of Taraxacum, 10 g of Radix Platycodonis , and 30 ml of White Vinegar.

2. A preparation method of traditional Chinese medicine microcapsules suitable for nasopharyngeal cancer tumor inhibitors is characterized in that it comprises the following steps:
1) Milk protein is used as the main wall material, and Artemisia annua starch is used as the auxiliary wall material. The auxiliary wall material accounts for 0-90% of the wall material mass;
the microcapsule preparation uses radix platycodonis oil as the core material;
2) The wall material is crushed to above 90 mesh and mixed evenly. Then, under the condition of stirring, slowly add the core material, after stirring and mixing uniformly, the formed core material is evenly dispersed in the inhibitor under stirring. After the wall material is fully hydrated, it is homogenized by a homogenizer at 20-40 MPa for 2 to 4 times to obtain a stable emulsion and complete the microcapsule embedding process;
3) The emulsion uses high temperature heat treatment, aldehyde cross-linking and enzymatic exchange methods to solidify the wall material protein, so that the microcapsule wall is insoluble in water, and the core material is solid or semi-solid in water;
4) The curing liquid is spray-dried to become a free-flowing solid powder.

3. The preparation method of traditional Chinese medicine microcapsules suitable for nasopharyngeal cancer tumor inhibitors according to claim 2, wherein the core material accounts for 55% of the mass of the microcapsules.
